(19) United States
(12) Patent Application Publication
(10) Pub. No.: US 2025/0017952 A1
(43) Pub. Date: Jan. 16, 2025

(54) ANTIFUNGAL COMPOUND, COMPOSITION AND USES THEREOF

(71) Applicants: SASTRA DEEMED UNIVERSITY, Thanjavur, Tamil Nadu (IN); VERSITECH LIMITED, Hong Kong (HK)

(72) Inventors: Prasanna NEELAKANTAN, Sai Ying Pun (HK); Adline Princy SOLOMON, Thanjavur (IN); Karthi SHANMUGAM, Thanjavur (IN)

(73) Assignees: SASTRA DEEMED UNIVERSITY, Thanjavur, Tamil Nadu (IN); VERSITECH LIMITED, Hong Kong (HK)

(21) Appl. No.: 18/717,355

(22) PCT Filed: Oct. 19, 2022
(86) PCT No.: PCT/IN2022/050932

§ 371 Date: Jun. 6, 2024

(30) Foreign Application Priority Data
Oct. 19, 2021 (IN) 202111018036

(51) Int. Cl. A61K 31/7076; A61P 31/10

(52) U.S. Cl. CPC A61K 31/7076; A61P 31/10

(57) ABSTRACT

The present invention relates to a compound for treating fungal disease, wherein said compound provide target-specific inhibition of fungal adhesion, biofilm formation, and filamentation without affecting their growth, at very low concentration. The compound of the present invention is selected from FDA approved drugs Cangrelor. Further, the present invention provides a composition comprising compound of the present invention and a pharmaceutically acceptable carrier. Further, the present invention provides a method of treating fungal infections using the compound and/or composition of the present invention. Furthermore, uses of the compound and/or composition is also being provided in the present disclosure.

### FIELD OF THE INVENTION

The present disclosure relates generally to the field of pharmaceuticals. Particularly, the present disclosure provides antifungal compounds, composition comprising the same and uses thereof. Further, the present disclosures also provide a method of treating fungal infections comprising the compounds and composition of the present disclosure.

### BACKGROUND OF THE INVENTION

Fungemia, a serious consequence of cytotoxic cancer chemotherapy, is characterized by fungal translocation through compromised mucosal barriers. By 2050, deaths attributable to drug-resistant infections will have reached 10 million per year. Drug resistance can be prevented by discovering and developing drugs (next-generation antifungals), that target fungal virulence, but not its growth or survival. The critical virulence aspects to deal with here, are biofilm formation and hyphal formation. The key fungi responsible for these infections is Candida albicans. Existing antifungal drugs are largely ineffective against Candida albicans biofilms. Higher concentrations that may be effective against biofilms are seriously toxic to the host (i.e., kidney or liver damage).

Having the status of being one of the most populous countries, India is estimated to have around 1 million cases of oropharyngeal candidiasis (Source: Google Medical Information-Apollo Hospitals, India). The studies report that HIV and cancer patients are more prone to this condition whose immune system is severely compromised (Maurya et al., 2013; Jayachandran et al., 2016). The incidence of antifungal resistance with respect to the Indian population is also reported (Chowdhary et al., 2018; Arun et al., 2019). With these alarming reports, there is considerable antifungal research in India that showed promising activity.

Antifungal discovery in India is largely focused on unraveling the potential of unknown Indian herbs and other Phyto molecules (Mehmood et al., 1999; Doddanna et al., 2013; Kamurthy et al., 2016; Latti et al., 2019; Murugesh et al., 2019; Sivareddy et al., 2019). With respect to the drug development against C. albicans, Indian researchers have identified inhibitors through in silico screening methodologies also. Rana et al. 2019 have repurposed Fluvastatin as CYP450 enzyme inhibitor of C. albicans. Another group identified Rrp9 as potential target for the development of anti-candida drug through in silico approaches (Ali et al., 2017). A recent work in the identification of potential targets through genome wide identification emphasizes the importance of C. albicans research worldwide (Verma et al., 2020). Studies by other groups have reported the advantages of discovering compounds that achieve biofilm and hypoallergenic inhibition without affecting growth, but further development was not achieved due to toxicity of their drugs (Fazly et al., 2013).

To address the problems existing in the art i.e., i) pressing need for novel antifungals that inhibit virulence and not growth; and ii) develop molecules with good safety profile, the present inventors have found a novel compound that can inhibit fungal biofilm formation and hyphal formation, without affecting its growth. It is well established that if the growth is not affected, the fungal cells cannot develop antimicrobial resistance to the molecule. Notably, most fungi are resistant to ALL the classes of antifungal drugs available in the market, including the most recent ones. There are NO commercial antifungals that can work using the same mechanisms as proposed by the present inventor.

There is, therefore, a need in the art, for: off-label applications of an FDA-approved compound where its safety profile is well established; and composition, mechanism of inhibiting C. albicans pathogenesis without affecting its growth, at very low concentration thereof, which overcome the drawbacks and shortcomings.

### SUMMARY OF THE INVENTION

The present invention relates to a compound for treating fungal disease, wherein said compound provide target-specific inhibition of fungal biofilm without affecting their growth, at very low concentration. The compound of the present invention is selected from FDA approved drugs Cangrelor. Further, the present invention provides a composition comprising compound of the present invention and a pharmaceutically acceptable carrier. Further, the present invention provides a method of treating fungal infections using the compound and/or composition of the present invention. Furthermore, uses of the compound and/or composition is also being provided in the present disclosure.

### BRIEF DESCRIPTION OF DRAWINGS

These and other features, aspects, and advantages of the present invention will become better understood when the following detailed description is read with reference to the accompanying drawings in which like characters represent like parts:

FIG. 1 illustrates crystal structure of C. albicans agglutinin-like sequence 3 (ALS3) complexed with hepta-threonine (PDB ID: 4LEB). ALS3 is shown as the surface and hepta-threonine is shown in cyan blue sticks. Peptide binding cavity (PBC) that is critical for binding are labeled with four-color codes. The yellow color code marked over the surface of the residues, 291-296 represents the binding cavity of ALS3 formed by BG2b. Similarly, the orange color denotes the BA2 that lies within the key residues, 167-173, whereas the loop A1-B1 are marked within the residues, 16-30 and a small β-strand containing the residue, K59 is denoted as red color. The residue of hydrophobic side chains, P29, F58, T61 and Y2 are represented in green color.

FIG. 2 illustrates stereo diagram showing ALS3 bound to the selected FDA approved drugs. The ALS3 molecule is shown as surface diagram whereas the key interacting binding site amino acids and the drug molecules are shown as sticks and the hydrogen bonds are shown in dotted black lines. Interactions of ALS3 with Cangrelor, are shown. Cangrelor made hydrogen bonding interaction with Lys59, Ser170, Val172 of BA2 strand Tyr 21 and Tyr 23 of A1B1 loop.

FIG. 3 illustrates antifungal and antibiofilm evaluation against C. albicans at varying concentrations: The effect of the Cangrelor on planktonic cells was determined by measuring the OD at 595 nm. A wide range of concentrations ranging from 100 μM-0.47 pM were considered for evaluation. Biofilm inhibitory effects were determined by measuring the biofilm biomass using the Crystal Violet (CV) assay. The dose-response curves were plotted by fitting the inhibition data using a 4-parameter logistic equation. Cangrelor inhibited biofilms at a minimum concentration of 120 pM

FIG. 4a illustrates CLSM analyses of biofilm inhibitory effects of Cangrelor against C. albicans SC5314. The images clearly demonstrate the biofilm structure, where the Cangrelor treated groups had loosely packed cells with predominantly green fluorescence (indicating live cells) and no biofilm formation, whereas the fluconazole treated cells were observed to have dead cells. The presence of red fluorescence in fluconazole treatment confirms its fungicidal effect. The COMSTAT analysis was done to quantify the biofilm coverage, average thickness, and biovolume from Z-stacks obtained from at least 5 regions of the glass slides, using a confocal laser scanning microscope (Olympus FLUOVIEW, FV1000) with a 40× objective lens (FIG. 4b).

FIG. 5 illustrates Cangrelor abolish C. albicans SC5314 biofilm. An overnight culture of C. albicans was grown overnight at 30° C. in YPD liquid media. C. albicans cells (1.5×10⁷ cells/ml) were seeded on the glass slides in the presence or absence of the ALS-3 drug-like chemotype, Cangrelor in YPD and incubated at 37° C. for 24 h. The representative scanned electron-micrographs show the analysis of 3 different spots in each of the triplicate experiments using image J software (a, d) Untreated C. albicans cells showing a dense network of hyphae, with more than 1200 cells (b.) Sub-inhibitory concentration of Cangrelor (120 pM) also inhibited biofilm formation and significantly reduced the number of cells to <56 (Inset). Statistical significance was determined using the unpaired Student's t test (P<0.0001).

FIG. 6 illustrates the phase contrast images of germ tube inhibition of Candida albicans for the three strains by cangrelor in a time-dependent manner till 6 h. The untreated C. albicans show the hyphal development from the 2nd hour, where the yeast cells adhere, and at the 6 h, the hyphal is elongated and branched. The yeast cells did not switch to the hyphal form in the cangrelor treatment.

FIG. 7 illustrates the phase contrast images of filamentation inhibition of Candida albicans by cangrelor after 24 h of inhibition. The untreated C. albicans showed filamentation forming a network after 24 h, while the Cangrelor treatment maintained the yeast form.

FIG. 8 illustrates the qRTPCR analysis of biofilm, hyphal and virulence genes in C. albicans biofilms treated with Cangrelor using 18srRNA as the housekeeping gene. Briefly, after 24 h incubation, planktonic cells were removed, and biofilm cells were collected, and RNA was extracted for the treated and untreated groups. Cangrelor downregulated the transcriptional regulators of biofilm and hyphal genes in addition to adhesin and hyphal specific protein. The data was obtained in triplicates and analysis was performed by the 2^{-ΔΔCt} method.

FIG. 9 illustrates the overall transcriptomic changes of Candida albicans pathogenesis (biofilm, hyphal formation, and drug inducing state in response to treatment with compound, Cangrelor (a) Hierarchical clustering heat map of 50 differentially expressed gene data. The color scale indicates the degree of correlation (green, high correlation; red, low correlation), while the height of the dendrogram branches represents the variability in gene expression between samples (under drug (Cangrelor) treated and untreated conditions) (b) Volcano plots showing significantly upregulated (red) and downregulated (blue) genes in samples (under drug (Cangrelor) treated and untreated conditions). A cutoff absolute value of log fold change >1 (2-fold change) was used. Adjusted P value <0.05 (c) Venn diagram illustrating the degree of overlap between various virulence genes (Biofilm Inducing (BI); Hyphal Inducing (HI); and Drug Resistance (DR) in C. albicans under drug (Cangrelor) treated and untreated conditions.

FIG. 10 illustrates the microscopic images of the monolayer T24 bladder epithelial cells infected with Candida albicans with and without Cangrelor treatment. In the control group, the C. albicans cells formed hypha, adhering and invading the epithelial cells, whereas cangrelor treatment inhibited hyphal morphogenesis. The yeast cells (circled in white) were maintained upon the treatment and thereby couldn't adhere and invade the epithelial cells.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps of the process, features of the invention, referred to or indicated in this specification, individually or collectively, and all combinations of any or more of such steps or features.

### Definitions

For convenience, before further description of the present disclosure, certain terms employed in the specification, and examples are collected here. These definitions should be read in the light of the remainder of the disclosure and understood as by a person skilled in the art. The terms used herein have meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article. The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference. The present disclosure is not to be limited in scope by the specific examples described herein, which are intended for the purposes of exemplification only. Functionally-equivalent products and methods are clearly within the scope of the disclosure, as described herein.

In an aspect of the present invention, a compound for treating fungal diseases has been provided, said compound provide target-specific inhibition of fungal adhesion, biofilm formation, and filamentation without affecting their growth at very low concentration; and wherein said compound is an FDA approved drug, Cangrelor.

In an embodiment, said fungal species is Candida albicans.

In another embodiment, said antifungal diseases are selected from oral and vaginal candidiasis.

In yet another embodiment, said compound has a concentration ranging from 7.6 pM to 120 pM.

In a preferred embodiment, said compound has a concentration of 120 pM.

In a second aspect of the present invention, a pharmaceutically acceptable topical formulation comprising the compound of the present invention has been provided along with pharmaceutically acceptable excipients.

In a third aspect of the present invention, a method of treating fungal diseases has been provided, wherein said method comprises the step of topically applying the topical formulation of the present invention, and wherein said formulation is applied in a therapeutic effective amount to a subject susceptible or suffering from fungal diseases selected from oral and vaginal candidiasis, said topical application inhibits fungal adhesion, biofilm formation, and filamentation of fungal species.

In a fourth aspect of the present invention use of an antifungal compound for treating fungal diseases selected from oral and vaginal candidiasis has been provided, wherein said compound is an FDA approved drug, Cangrelor.

An off-label topical application of non-toxic FDA-approved drug, Cangrelor for treating candidiasis (Oral/Vaginal) is disclosed. Said FDA-approved drug, Cangrelor is screened against the Candida albicans cell wall protein, ALS3 (PDB ID: 4LEB) using High throughput virtual screening (HTVS) from in-house FDA approved drug database. Said Cangrelor made hydrogen bonding interaction with Lys59, Ser170, Val172 of βA2 strand Tyr 21 and Tyr 23 of A1B1 loop of ALS3.

The disclosed anti-fungal compound, Cangrelor, and the method offer at least the following advantages and effects:
inhibited biofilms at a very low concentration of 120 pM without affecting growth;
germ tube inhibition of Candida albicans in a time-dependent manner till 6 h;
No transition to hyphal form was observed in the Cangrelor treatment; and
Said compound impacted on the pathogen-host (monolayer T24 bladder epithelial cells) adherence and invasion ability and offer superior safety profiles.

The disclosed anti-fungal compound, Cangrelor showed an overall transcriptomic change in the genes that regulates biofilm formation, hyphal formation, and drug resistance to azoles.

The current clinical use of the disclosed Cangrelor is via., systemic route and our future invention provides a novel pharmaceutically acceptable topical formulation to solve the health risk of Candidiasis (Oral & Vaginal).

The disclosed drug effect on the Candida albicans pathogenesis are as follows

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

### Example 1

### Target Identification and High Throughput Virtual Screening:

The concept of drug repositioning has resulted in a paradigm shift in the drug development process as it harnesses the idea of finding new dimensions to existing approved drugs (Pushpakom S et al., 2018). High throughput screening (HTS) using biological and biochemical assays have been traditionally followed to identify the new use to an approved drug. Time consumption, huge expense and increased false positives of HTS has led to the computational alternative of virtual screening (Pyzer-Knapp Eo et al., 2015; Subramaniam S et al., 2008). HTVS overcomes the shortcomings of HTS in terms of significant reduction in time scale, costs and the expansion of screening large compound libraries to discover lead molecules with target-specific activity (Ma D L et al., 2013).

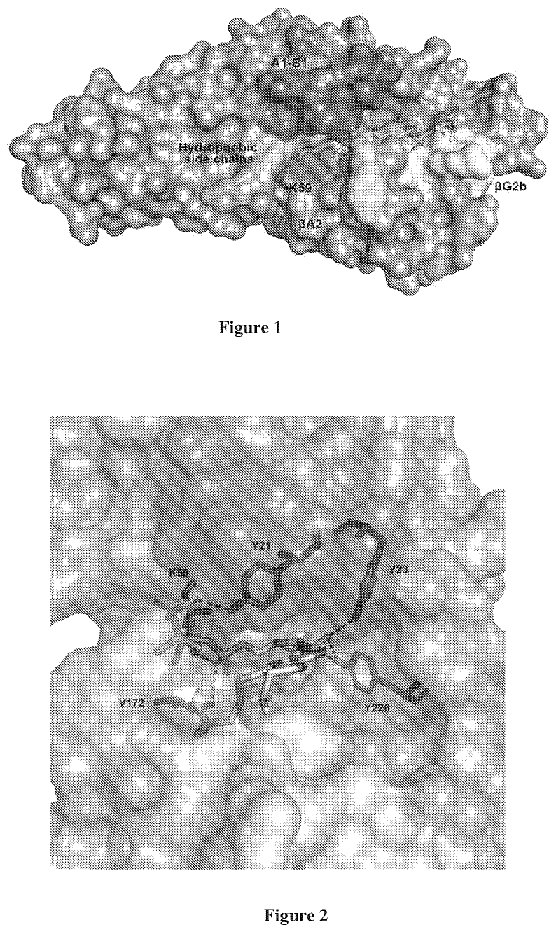

In a structure based virtual screening process, target identification plays a vital role and serves as the primary step. The present inventors selected the ALS3 adhesin of C. albicans as the potential target to screen FDA approved drugs obtained from Drug Bank Database (Wishart D S et al., 2018). The crystal structure of the Peptide Binding Cavity (PBC) of ALS3 with PDB ID-4LEB was chosen for the study (Lin J et al., 2014). It was established previously that the N-terminal domain is responsible for the adhesive activity towards different substrates (Liu Y and Filler S G, 2011; Bamford C V et al., 2015). 4LEB has the resolution of 1.4 Å having sequence length of 300 amino acids and complexed with hepta-threonine. Functionally, studies have shown that PBC is vital for the adhesive function of ALS3. Structurally, Lys59 is an important binding cavity residue of PBC. The peptide binding cavity of ALS3 comprises of BG2b strand with residues 291-296, βA2 strand with residues 167-173, A1-B1 loop having residues 16-30 and a K59 present as small β-strand. The hydrophobic chains comprise P29, F58, T61 and Y298 (FIG. 1). Hence, the compounds which made interactions with these β sheets and more importantly with Lys59 were selected for further investigations.

A total of 2482 FDA approved drugs were screened for ALS3 inhibition using the Schrodinger GLIDE docking panel (Halgren R A et al., 2004). HTVS is achieved by ligand positioning refined by torsional flexible energy optimization which are further refined by Monte Carlo sampling. Top 50%, 25%, 15% of the molecules were chosen for HTVS, SP and XP analysis, respectively (Chen Y-C, 2015). Finally, the hit molecules were ranked based on the protein specific conformation.

Combining the aspects of binding score as well as the specific pose, XP docking mode is considered accurate (Friesner R A et al., 2006). Hence the promising compound that made essential interactions with ALS3 PBC and had a promising XP dock score were chosen for further investigations (Table 1). The stability of the docked poses was tested by calculating the binding energy with Prime-MMGBSA (Jacobson M P et al., 2004). This analysis revealed a 4G binding energy >−40 Kcal/mol for Cangrelor which confirmed the stability of the docked pose.

The interactions with the key residues play an important role in the intended activity. In the case of ALS3, an invariant Lys59 (conserved residue of ALS family), is important for the initial binding of the peptide and for the orientation of the peptide ligand in the PBC. Additionally, the β sheets-βG2b and A2 are essential for the stable interaction of the ligands with PBC (Lin J et al., 2014). Additional hydrogen bonding with the key residues on the binding pocket and hydrophobic interactions were observed, which established the stability of these interactions.

In this regard, the minimum binding energy of −53.87 kcal/mol was obtained for Cangrelor which is supported by the stable hydrogen and hydrophobic interactions. Hydrogen bonding was established with Val172 of βA2 strand, Tyr21, Tyr23 of A1B1 loop along with Lys59 and Tyr226. Hydrophobic interactions with Trp295 and Thr296 of G2β strand were observed.

While a previous study reinforced the importance of π-πinteractions between the ligand and PBC (Kioshima E S et al., 2019), this was not observed in any of the drugs screened in this study. FIG. 2 shows the detailed 3D interaction map of the Cangrelor with Peptide Binding cavity of ALS3. Hence, based on the detailed docking analysis including binding energy, and crucial interactions-Cangrelor, were investigated for their effects in vitro.

TABLE 1
Summary of the interaction of FDA approved drug
Cangrelor and the target, ALS3 binding site

| | | Glide | MMGBSA | | | |
|---|---|---|---|---|---|---|
| | | X [symbol] | ΔG bind | Hydrogen Bond | | Hydroph [symbol] |
| Drug ID | Drug Name | Score | (kcal/mol) | Interaction | Distance | Interacti [symbol] |
| DB06441 | Cangrelor | −11.919 | −53.87 | Tyr21A OH—OP Lig | 1.58 | Trp295A |
| | | | | Tyr23A OH—OH Lig | 2.80 | Thr296A |
| | | | | Lys59A NH—OP Lig | 1.92 | |
| | | | | Val172A NH—OP Lig | 2.16 | |
| | | | | Tyr226A HO—HO Lig | 1.77 | |
| | | | | Tyr226A HO—HO Lig | 2.00 | |

[symbol]

indicates data missing or illegible when filed

### Example 2

Top Hit Drug Inhibits C. albicans Biofilm without Affecting the Planktonic Growth

ALS3 is a hyphal specific adhesin and is not vital for the survival of C. albicans (Shinobu-Mesquito C S et al., 2020). Thus, the compounds that are intended to target ALS3 should not inhibit the fungal growth. The importance of ALS3 in the biofilm formation and maturation is well established. Hence, the present inventors evaluated the growth and biofilm inhibitory role of the Cangrelor. The growth of C. albicans was not inhibited for the Cangrelor, establishing its role as anti-virulence compounds and not anti-fungal. The biofilm inhibitory effect was clearly demonstrated for Cangrelor (FIG. 3). Cangrelor exhibited biofilm inhibition at the BIC₅₀ (50% Biofilm Inhibition Concentration) was recorded at 120 pM. Previous study reports that the Cangrelor was repurposed as anti-aging (Mofidifars et al., 2018) and antiviral (Mucke H A M, 2017) agents. Based on the in silico and in vitro antibiofilm assay, the present study extends the spectrum of FDA drug, Cangrelor by specifically studying the high-value target namely biofilm formation, to establish the anti-virulence effects of these test compounds against C. albicans.

### Example 3

Cangrelor Reduce C. albicans Biofilm Formation.

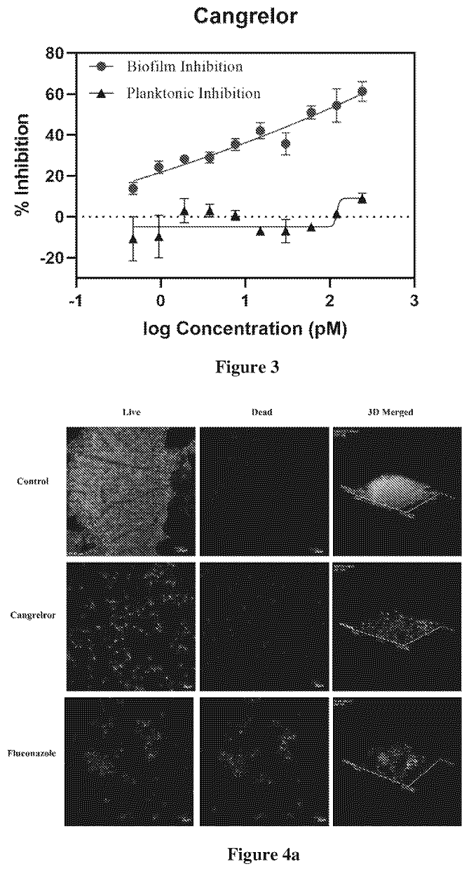

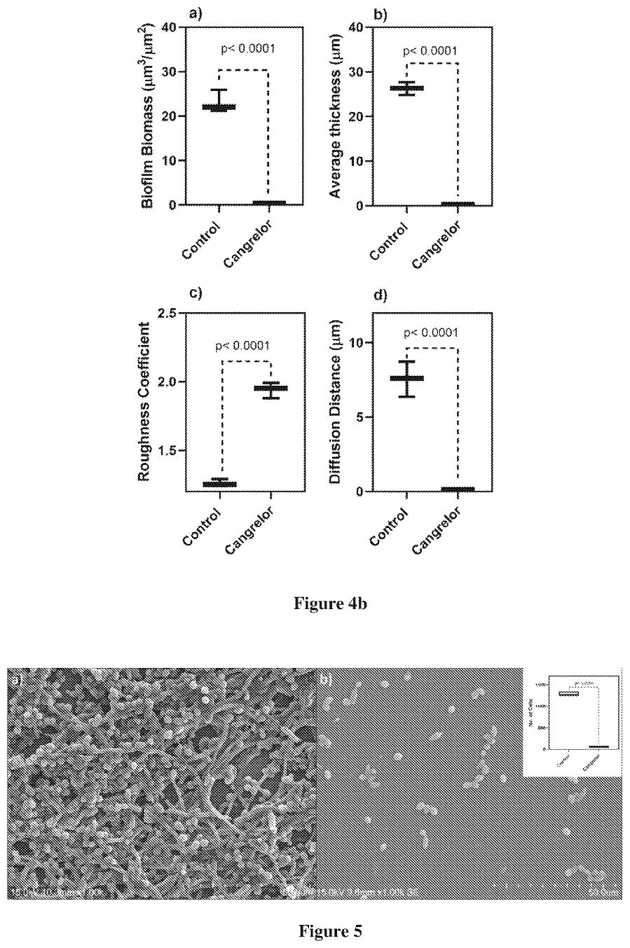

ALS3 is strongly associated with the initiation and formation of a robust three-dimensional biofilm architecture. FIG. 4 reveals the biofilm architecture of the untreated and treated biofilms. CLSM images are in accordance with the microtiter plate biofilm inhibitory assays. The anti-adherence property is expanded to anti-biofilm property of the screened test compounds. Further quantitative analysis using COMSTAT revealed a significant reduction in the biomass for the treatment groups. While a biofilm biomass of 25.96 μm was recorded for the untreated biofilm, Cangrelor. The average biofilm thickness reduced from 28.64 μm for control to 0.5 μm for Cangrelor. The other physical parameters such as increased roughness coefficient from 1.2 (control) to 1.91 (treatment) and reduced diffusion distances from 8.73 μm to 0.18 μm confirmed the architectural changes in the treated biofilms. Such physical parameters define the intact architecture of the biofilm formed. Roughness coefficient defines the heterogeneity of the biofilm (Givskov M et al., 2000), untreated control biofilm is less heterogenous with uniform biomass whereas the increased coefficient in the treatment groups establishes the heterogenous distribution of biofilm (reduced and uneven biofilm biomass). Diffusion distances explains the solute movement in the biofilm (Stewart P S, 2003), the reduced diffusion distance in the treatment group describes the perturbed biofilm which can lead to the easy clearance of biofilm by the host cells. The biofilm inhibition of the test compound was further confirmed using SEM analysis. FIG. 5 shows the scanning electron micrographs of treated and untreated C. albicans grown in YPD. Cangrelor (120 pM) abolished filamentation when compared to the untreated control. While the untreated control had more than 1200 cells, Cangrelor treatment resulted in <60 cells. The qualitative analysis of SEM corroborates well with the CLSM analysis and with the semi-quantitative data. It is interesting to note that the C. albicans cells of the treatment groups were in yeast forms whereas the untreated control was found to be in filamentous form. ALS3 is established as the hyphal specific protein, which is associated with the important virulence characteristics like biofilm formation, maintenance, and ferritin binding. Through these studies, the present inventors prove that the test compounds are inhibiting the initial adhesion of the cells which has led to the reduced biofilm formation. The reduction in the number of C. albicans cells indicates the reduced biofilm biomass and reduced biofilm maintenance.

### Example 3

### Cangrelor Inhibits Filamentation in a Time-Dependent Manner

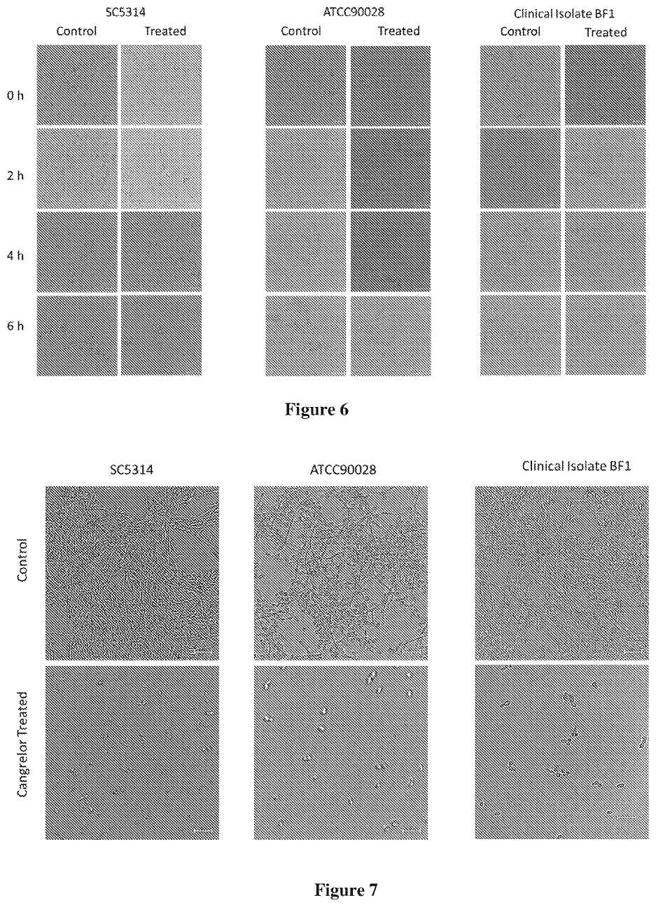

A time-dependent mapping of the hyphal development was done until 6 h upon Cangrelor treatment and compared to the untreated control for the three strains of C. albicans, including the standard strains-SC5314 and ATCC90028, and clinical isolate, BF1. FIG. 6 shows that the treatment inhibits the hyphal morphogenesis from the initial incubation time and is maintained even at 24 h (FIG. 7). The same trend was seen in all three strains considered. The morphogenesis switch inhibition is essential as the primary virulence characteristic of C. albicans is hyphal formation. The hyphal form is necessary for biofilm formation and polymicrobial interaction to invade the host cell and overcome the immune response. Hence, Cangrelor inhibits the morphogenetic switch

### Example 4

### Downregulation of Virulence Associated Genes by Cangrelor.

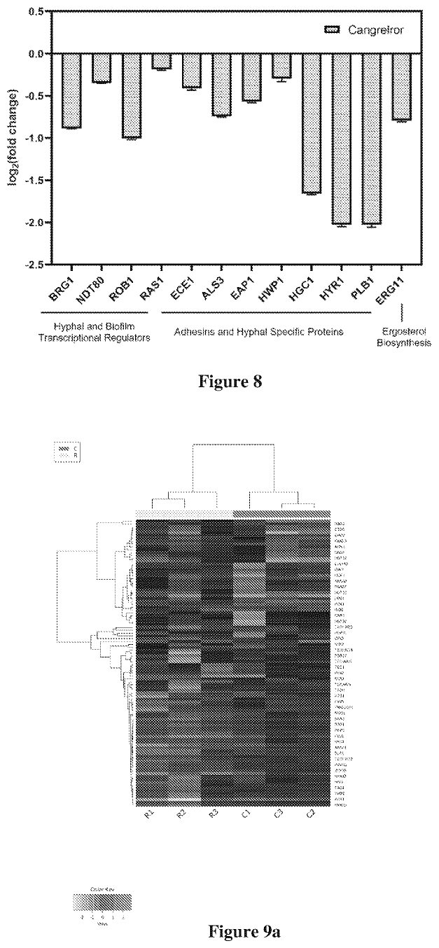

Previous studies have clearly demonstrated the upregulation of ALS3 in C. albicans biofilm cells than in planktonic cells (Samaranayake Y H et al., 2013). The genes that are responsible for biofilm formation, adhesion, hyphal formation and ergosterol synthesis were considered for the analysis as seen in FIG. 8. Cangrelor (240 pM) treatment downregulated the adhesion and invasion genes.

It is established that ALS3 is a hypha-specific gene and is expressed only in pseudo hyphae and hyphal forms of C. albicans (Liu Y and Filler S G, 2011). The present inventors investigated the expression of biofilm master regulators, RAS, NDT80, ROB1 and BRG1 upon the treatment of Cangrelor. Studies have shown that these master regulators are specific for biofilm formation and have no role in the growth process (Mc Call A D et al., 2018). Thus, the downregulation of these regulators implies the impairment in biofilm formation specifically. In addition, the adhesin genes—ALS3, HWP1 (Hyphal cell wall mannan protein), HYR1 (Hyphal Regulated gene), and EAP1 (Cell-cell adhesin) were considered. It was shown that the adhesin genes play a role in microcolony formation (Mc Call A D et al., 2018). Studies have shown that these transcriptional regulators have a direct effect on the expression of hyphal specific genes. For instance, BRG1, ROB1 and NDT80 regulate the expression of ALS3, HYR1, EAP1, ECE1, and HWP1 (Samaranayake Y H et al., 2013). Thus, the downregulation of transcriptional regulators by Cangrelor have downstream effect on the hyphal specific genes. The phenotypic observation of reduction in biofilm correlates with the downregulation of the genes responsible for hyphal proteins and biofilm regulatory factors.

It has been shown previously that ALS3 and HWP1 complement each other in the event of biofilm formation (Liu Y and Filler S G, 2011). Downregulation of both these genes in addition to other genes such as HGC1 confirms the anti-adherence and anti-biofilm property of the tested compounds. In addition, gene encodes for the invasin phospholipase, PLB1, was downregulated ˜2 log fold, demonstrating the interference in the fungal invasion (Mayer F L et al., 2013), and further research is required on organotypic models to confirm the phenotypic effects. The morphological transition of C. albicans results in filamentous hyphae that secrets candidalysin, a 31-amino acid peptide toxin required for virulence factors such as adhesins, biofilm formation and filamentation (Richardson J P et al., 2018; Anis E et al., 2020). Candidalysin is a cytolytic peptide encoded by ECE1 gene and is required for successful infections (Richardson J P et al., 2018). The downregulation of ECE1 gene by Cangrelor might contribute to their anti-virulence property. ERG11 has been demonstrated to be the principal mechanism involved in the development of drug resistance to azole antifungals by C. albicans (Riberiro M A and Paul C R, 2007). This notable finding that Cangrelor downregulated the ERG11 gene indicates the poor possibility of development of resistance to these compounds.

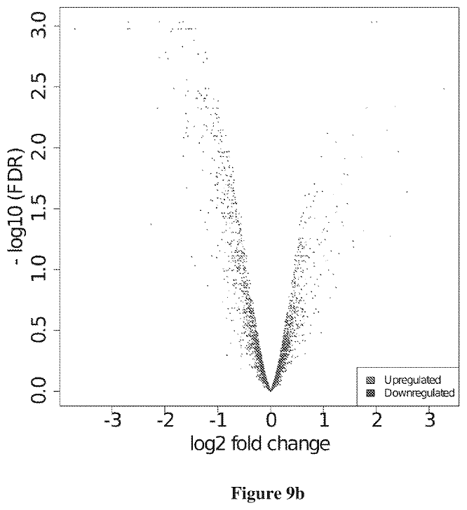

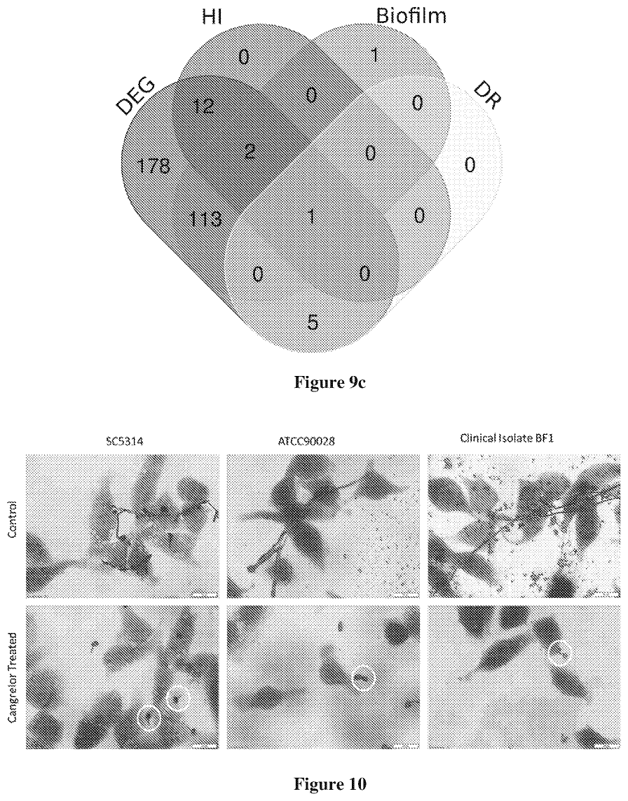

In the RNAseq data analysis (FIG. 9a, b, c) of the top 311 genes differential expression in response to the compound Cangrelor treated/untreated state in C. albicans, various virulence genes inducing biofilm, hyphae and drug-resistance were observed to be downregulated. Most notably, the biofilm master regulator genes such as RAS, NDT80. TRY6, FYV5 (Pentland et al., 2018; Murillo et al., 2005; Nett et al., 2009a), drug-resistance genes MAK1 and RPP1 (Nett et al., 2009b; Levitin & Whiteway, 2007a) and upregulation of the genes, GAL and MAF1 that negatively regulates (repressors) the biofilm phenotype in C. albicans (Nobile et al., 2012). Overall, more than 90 biofilm inducing genes are found to be downregulated in drug (Cangrelor) induced state and it correlates with the inventor's phenotypic assays. In Candida albicans, the potential shift from yeast to hypha is a route cause to host invasion and symbiotic relationship with other pathogens, where the data was significant to show downregulation of hyphal-specific genes such as ECE1, RAS, NDT80 (Moyes et al., 2016). Along with the Candidalysin encoding gene ECE1, there are several other drug (azole) resistances inducing genes, PDR16, UTP22, PGA42 and ENP2 are observed to be downregulated with an adjusted p-value cutoff of 0.05. This observation was very crucial to the inventor's claim that the compound, Cangrelor could potentiate the anti-fungal susceptibility (Moyes et al., 2016; Maglott et al., 2007b; Levitin & Whiteway, 2007b; Chaudhuri et al., 2011; Singh et al., 2011). Taken together, Cangrelor systematically downregulate the virulence features to potentially curb the pathogenesis of C. albicans.

### Example 5

Cangrelor Inhibits the Cell Adhesion and Invasion of C. albicans in the Monolayer of Epithelial Cells

Previous results established the role of Cangrelor in efficiently inhibiting hyphal morphogenesis. The ability of Cangrelor to maintain the yeast form in bladder epithelial cell lines was shown. FIG. 10 shows the establishment of hyphal adhering to the epithelial cells and also invading the cells in the absence of treatment. In the case of Cangrelor treatment, the cells were maintained in the yeast form, thereby reducing adherence and invasion. The results corroborate the previous in vitro and gene expression studies, which proved that Cangrelor could retain yeast form and inhibit the virulence process of C. albicans.

1. A compound for treating fungal diseases, wherein said compound provide target-specific inhibition of fungal adhesion, biofilm formation, and filamentation without affecting their growth at very low concentration; and wherein said compound is an FDA approved drug, Cangrelor.

2. The compound as claimed in claim 1, wherein said fungal species is Candida albicans.

3. The compound as claimed in claim 1, wherein said antifungal diseases are selected from oral and vaginal candidiasis.

4. The compound as claimed in claim 1, wherein said compound has a concentration ranging from 7.6 pM to 120 pM.

5. The compound as claimed in claim 1, wherein said compound has a concentration of 120 pM

6. A pharmaceutically acceptable topical formulation comprising the compound as claimed in claims 1 to 5 along with pharmaceutically acceptable excipients.

7. A method of treating fungal diseases, wherein said method comprises the step of topically applying the topical formulation as claimed in claim 6, and wherein said formulation is applied in a therapeutic effective amount to a subject susceptible or suffering from fungal diseases selected from oral and vaginal candidiasis, said topical application inhibits fungal adhesion, biofilm formation, and filamentation of fungal species.

8. Use of an antifungal compound for treating fungal diseases selected from oral and vaginal candidiasis, wherein said compound is an FDA approved drug, Cangrelor.
